(19) United States
(12) Patent Application Publication
(10) Pub. No.: US 2015/0056288 A1
(43) Pub. Date: Feb. 26, 2015

(54) MODIFIED RELEASE LIQUID PHARMACEUTICAL COMPOSITION COMPRISING BROMOPHENIRAMINE, PSEUDOEPHEDRINE AND DEXTROMETHORPHAN

(71) Applicant: WOCKHARDT LIMITED, Aurangabad 0 (IN)

(72) Inventors: Vivek Dubey, Hyderabad (IN); Rahul Dabre, Raigad (IN); Girish Kumar Jain, Mumbai (IN)

(73) Assignee: WOCKHARDT LIMITED, Aurangabad 0 (IN)

(21) Appl. No.: 14/363,795

(22) PCT Filed: Nov. 1, 2012
(86) PCT No.: PCT/IB2012/056087

§ 371 Date: Jun. 7, 2014

(30) Foreign Application Priority Data
Dec. 14, 2011 (IN) 3510/MUM/2011

(51) Int. Cl. A61K 47/10; A61K 31/135; A61K 31/4402; A61K 31/485

(52) U.S. Cl. CPC A61K 47/10; A61K 31/485; A61K 31/135; A61K 31/4402
USPC 424/490; 514/289

(57) ABSTRACT

There is provided a modified release liquid pharmaceutical composition comprising combination of dextromethorphan, bromrpheniramine and pseudoephedrine or pharmaceutically acceptable salts thereof. The invention further provides process for preparation of such compositions.

### FIELD OF THE INVENTION

The present invention relates to a modified release liquid pharmaceutical composition comprising combination of brompheniramine, pseudoephedrine and dextromethorphan or pharmaceutically acceptable salts thereof. The invention further provides process for preparation of such compositions.

### BACKGROUND OF THE INVENTION

Upper respiratory symptoms include symptoms such as nasal congestion, sinusitis, cough, cold, cold-like symptoms, allergic rhinitis resulting from a cold or influenza infection or allergic reactions, upper respiratory mucosal congestions such as those seen in perennial and allergic rhinitis. Eustachian tube congestion, runny nose, post nasal drip are the most common ailments which are frequently seen in individuals. Though the ailments generally are not life threatening, result in severe discomfort and hamper day-to-day working of the individuals.

The symptoms are treated using variety of therapeutic agents such as antihistamines, decongestants, cough suppressants or antitussives, expectorants and preferably combinations thereof. Several attempts have been made to develop compositions comprising combination of the said therapeutic active agents in different dosage forms.

Dextromethorphan is marketed as dextromethorphan hydrobromide and dextromethorphan polistirex. Chemically dextromethorphan hydrobromide is a salt of the methyl ether of the dextrorotatory isomer of levorphanol. It is chemically designated as 3-methoxy-17-methyl-9α,13α,14α-morphinan hydrobromide monohydrate with the following structural formula I:

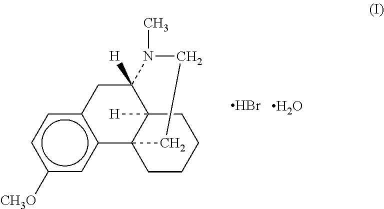

Dextromethorphan polistirex (dextromethorphan hydrobromide complexed with resin) is marketed under the trade name Delsym® by Reckitt Benckiser in the form of extended release suspension indicated for the treatment of non-productive cough.

Pseudoephedrine is marketed as pseudoephedrine hydrochloride and pseudoephedrine sulfate. Pseudoephedrine hydrochloride, is chemically [S—(R*,R*)]-α-[1-(methylamino)ethyl]-benzenemethanol hydrochloride having the structural formula (II):

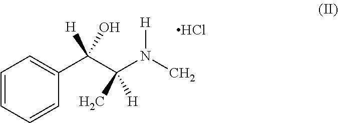

Pseudoephedrine hydrochloride is marketed as extended release under the trade name “Sudafed 24 Hour®” by Alza indicated for nasal and sinus congestion. Pseudoephedrine is available in different dosage forms including tablet, extended release tablet, capsule and suspension as a decongestant medication.

Brompheniramine was marketed as Brompheniramine maleate under the trade name DIMETANE-DX® in the form of syrup by Robins AS and DIMETANE tablet and extended release tablets marketed by Wyeth. Chemically Brompheniramine is γ-(4-Bromophenyl)-N,N-dimethyl-2-pyridinepropanamine with the structural formula (III).

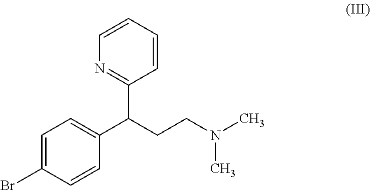 [SMILES: CN(C)CCC(c1ccc(Br)cc1)c1ccccn1]

Liquid formulations for oral delivery of pharmaceutical agents are desirable because certain patients, such as children and the elderly, are unable to swallow capsules or tablets.

Liquid formulations comprising brompheniramine, pseudoephedrine and dextromethorphan are available over-the-counter under the brand name of Bromfed DM and Dimetane DX. Bromfed DM contains Brompheniramine maleate, pseudoephedrine hydrochloride, dextromethorphan hydrobromide and alcohol. Bromfed DM is indicated for the treatment of the symptoms of the common cold and allergic rhinitis, such as runny or stuffy nose, cough, itchy or watery eyes and sneezing. The dosing schedule includes administration of 2 teaspoonfuls every 4 hours i.e. 10 ml of the syrup has to be administered every 4 hours.

Dimetane DX contains 2 mg Brompheniramine maleate, 30 mg pseudoephedrine hydrochloride, 10 mg dextromethorphan hydrobromide. As per the dosing schedule of Dimetane DX two teaspoonfuls has to be administered every 4 to 6 hours. The total dose should not exceed 12 teaspoonfuls in 24 hour period.

The commercially available cough syrups of Bromfed or Dimetane DX has to be administered 4-6 times a day. This often leads to non-compliance of patient to the treatment. Thus, there is a need for the development of compositions comprising effective amounts of brompheniramine, pseudoephedrine, dextromethorphan which can provide sustained effect and relief from the symptoms of upper respiratory tract infections for longer period even by reducing the frequency of administration of the composition in order to improve the patient compliance and adherence to the treatment.

Several attempts have been made to develop compositions comprising combination of the said therapeutic active agents in different dosage forms. The use of tannate suspensions for pharmaceutical use is well-known. However, the tannate has a complex, non-uniform chemistry and astringent properties. The tannate salt complex of the active agent is a significantly larger molecule contributing to bigger size of the dosage form. Further many antihistamine tannates are heat sensitive and therefore undergo decomposition quite readily upon prolonged exposures to temperatures as low as 50° C. These problems could cause significant dosing and processing problems during manufacture and increase the likelihood that commercially available pharmaceutical products contain variable and in some instances sub-therapeutic levels of said active drug substances.

Brompheniramine, pseudoephedrine and dextromethorphan all are bitter and unpleasant tasting drugs. Dextromethorphan has along with bitter taste an un-aesthetic mouth-feel and an unpleasant after-taste. In order to ensure better patient compliance bitterness masking becomes essential. Thus, there is a great need for developing a taste-masked composition, the administration of which would minimize the occurrence of adverse events and improve patient compliance, encouraging patient's adherence to the prescribed dosing regimen. An ideal composition should have good tasting presentation to achieve higher patient compliance.

U.S. Pat. No. 4,999,189 discloses a sustained release oral pharmaceutical composition comprising a drug-resin complex suspended in a liquid carrier.

U.S. Pat. No. 5,980,882 discloses a pharmaceutical composition comprising a drug-resin complex and a chelating agent in the form of a solid or a gel.

European Patent No. 0946145B1 discloses a sustained release pharmaceutical composition comprising 20% to 80% of the drug/resin complexes coated with a water-permeable diffusion barrier.

U.S. Pat. No. 6,001,392 discloses a sustained release pharmaceutical composition comprising a drug/resin complex including a coated portion that comprises about 20 to about 80% of the drug/resin complex, and an uncoated portion.

U.S. Patent Application No. 20070092553 discloses a taste-masked pharmaceutical composition comprising a drug-resin complex and a highly compressible, free-flowing pharmaceutical excipient.

U.S. Patent Application No. 20080118570 discloses a method of making a coated drug/resin complex that comprises providing a drug/resin complex having an outer surface and comprising a plurality of resin beads and a therapeutically effective drug component.

U.S. Pat. No. 4,221,778 discloses a pharmaceutical preparation comprising ion exchange resin particles having a pharmacologically active drug absorbed thereon to form drug-resin complex particles.

U.S. Pat. No. 5,186,930 discloses a stable sustained release wax- and polymer-coated drug-ion exchange resin complexes especially useful in preparing oral suspensions.

European Patent No. 0139881B1 discloses a pharmaceutical composition containing a coated drug-resin complex suspended in a liquid carrier, and chlorpheniramine as uncoated resin complex.

U.S. Pat. No. 6,869,618 discloses a manufacturing process for the preparation of liquid or semi-solid dosage forms containing a tannate salt complex of active pharmaceutical ingredients.

U.S. Pat. No. 7,101,572 discloses a substantially taste masked aqueous liquid pharmaceutical composition that contains an otherwise unpleasant tasting drug.

U.S. Pat. No. 4,996,047 discloses oral controlled-release pharmaceutical preparations comprising drug-ion-exchange resin complex.

U.S. Pat. No. 6,509,492 discloses liquid suspension comprising pseudoephedrine tannate, chlorpheniramine tannate and dextromethorphan tannate.

U.S. Pat. No. 6,790,980 discloses pharmaceutical liquid suspension of tannate therapeutic agents such as dexchlorpheniramine, chlorpheniramine, pseudoephedrine, dextromethorphan.

U.S. Pat. No. 5,980,882 discloses pharmaceutical composition comprising a drug-resin complex and a chelating agent.

U.S. Pat. No. 7,094,429 discloses a process of preparing tannate salt complex of an antihistamine, a decongestant, an antitussive or anticholinergic.

U.S. Pat. No. 5,196,436 discloses antitussive composition for peroral administration consisting dextromethorphan and orally-acceptable pharmaceutical carrier in the form of an aqueous-based liquid, or solid dissolvable in the mouth.

U.S. Patent Application No. 20060121066 discloses a pharmaceutical composition comprising sucralose to mask a bitter taste of any active ingredients.

U.S. Application No. 20050232993A1 discloses pharmaceutical dosage form comprising an antihistaminic drug and one second drug selected from decongestants, antitussives, expectorants, mucus thinning drugs, analgesics and antihistamines, both having different plasma half-lives.

In spite of the several attempts made in the art for preparing compositions comprising combination of therapeutic agents, there still exists a continuing need of a taste-masked liquid composition comprising a triple combination of brompheniramine, pseudoephedrine and dextromethorphan, which can exhibit modified release of these active agents.

The present inventors have developed a pharmaceutical liquid composition comprising a triple combination of brompheniramine, pseudoephedrine and dextromethorphan or pharmaceutically acceptable salts thereof for the treatment of said ailments wherein the composition is in the form of liquid composition capable of providing sustained therapeutic effect for 8 to 24 hours upon administration, and thus may obviate the need of frequent dose administration.

Pharmaceutical composition of the invention not only exhibit modified release characteristics that reduce the number of administrations required to maintain consistent blood levels of said active agents, but also display excellent palatability and stability as a suspension.

### SUMMARY OF THE INVENTION

In one general aspect of the invention, there is provided a modified release liquid pharmaceutical composition comprising:
a. brompheniramine or pharmaceutically acceptable salt thereof;
b. pseudoephedrine or pharmaceutically acceptable salt thereof;
c. dextromethorphan or pharmaceutically acceptable salt thereof complexed or coated with an ion exchange resin,
   wherein the composition exhibits steady therapeutic levels over 8 to 24 hours after administration.

In another general aspect of the invention, there is provided a modified release liquid pharmaceutical composition comprising:
a. brompheniramine or pharmaceutically acceptable salt thereof complexed or coated with an ion exchange resin;
b. pseudoephedrine or pharmaceutically acceptable salt thereof;
c. dextromethorphan or pharmaceutically acceptable salt thereof complexed or coated with an ion exchange resin,
   wherein the composition exhibits steady therapeutic levels over 8 to 24 hours after administration.

In another general aspect of the invention, there is provided a modified release liquid pharmaceutical composition comprising:
a. brompheniramine or pharmaceutically acceptable salt thereof complexed or coated with an ion exchange resin;
b. pseudoephedrine or pharmaceutically acceptable salt thereof complexed or coated with an ion exchange resin;
c. dextromethorphan or pharmaceutically acceptable salt thereof complexed or coated with an ion exchange resin,
   wherein the composition exhibits steady therapeutic levels over 8 to 24 hours after administration.

In another general aspect of the invention, at least one of bromrpheniramine, pseudoephedrine, dextromethorphan or their pharmaceutically acceptable salt is complexed or coated with one or more resins.

In another general aspect of the invention, at least one of bromrpheniramine, pseudoephedrine, dextromethorphan or their pharmaceutically acceptable salt is complexed or coated with polistirex.

In another general aspect of the invention, there is provided a modified release liquid pharmaceutical composition comprising dextromethorphan polistirex, brompheniramine polistirex, and pseudoephedrine polistirex along with one or more rate controlling polymers and pharmaceutically acceptable excipients, wherein the composition provides steady therapeutic levels over 8 to 24 hours after administration.

In another general aspect of the invention, there is provided a modified release liquid pharmaceutical composition comprising dextromethorphan polistirex; brompheniramine polistirex, and pseudoephedrine polistirex along with one or more rate controlling polymer and pharmaceutically acceptable excipients, wherein the composition provides steady therapeutic levels over 12 hours after administration.

In another general aspect of the invention, there is provided a method for treating the symptoms of upper respiratory tract infection by administering a modified release liquid pharmaceutical composition comprising dextromethorphan, brompheniramine, and pseudoephedrine or pharmaceutically acceptable salt thereof, wherein at least one of bromrpheniramine, pseudoephedrine, dextromethorphan or their pharmaceutically acceptable salt is complexed or coated with polistirex, and characterized in that the composition provides steady therapeutic levels over 8 to 24 hours after administration, to a patient in need thereof.

In another general aspect of the invention, there is provided a method for treating the symptoms of common cold and allergic rhinitis by administering a modified release liquid pharmaceutical composition comprising dextromethorphan, brompheniramine, and pseudoephedrine or pharmaceutically acceptable salt thereof, wherein at least one of bromrpheniramine, pseudoephedrine, dextromethorphan or their pharmaceutically acceptable salt is complexed or coated with polistirex, and characterized in that the composition provides steady therapeutic levels over 8 to 24 hours after administration, to a patient in need thereof.

While the present invention has been described in terms of its specific embodiments, certain modifications and equivalents will be apparent to those skilled in the art and are intended to be included within the scope of the present invention.

### DETAILED DESCRIPTION OF THE INVENTION

Drug-resin complexes have several advantages over pure drugs in ordinary formulations. Many drugs are bitter and some smell bad. Getting a patient, particularly a small child or an elderly person, to swallow something that tastes or smells bad can be a serious problem. Complexing such a drug with a resin often improves the taste or the smell. Complexing a drug with a resin can also change its physical characteristics. This change may make the drug more convenient to mass produce or easier for patients to take.

“Modified release dosage forms” are defined by the USP as those whose drug release characteristics of time course and/or location are chosen to accomplish therapeutic or convenience objectives not offered by conventional forms. The USP considers that the terms controlled release, prolonged release and sustained release are interchangeable with extended release. Accordingly, the terms “modified-release”, controlled-release”, “prolonged-release”, “extended-release”, and “sustained-release” are used interchangeably herein.

Dextromethorphan polistirex acts as an antitussive in suppressing the cough reflex in the medulla. Treatment is intended to relieve cough frequency without abolishing protective cough reflex. Polistirex is an edible ion exchange resin that forms complex with the medicinally active agents.

Brompheniramine polistirex is a potent, short-acting antihistamine. It competes with histamine for H1-receptor sites on effector cells in the gastrointestinal tract, blood vessels, and respiratory tract and substantially diminishes or abolishes the action of histamine in the body.

Pseudoephedrine is an orally active sympathomimetic amine and exerts a decongestant action on the nasal mucosa. Pseudoephedrine sulfate is recognized as an effective agent for the relief of nasal congestion due to allergic rhinitis. Pseudoephedrine produces peripheral effects similar to those of ephedrine and central effects similar to, but less intense than, amphetamines.

As used herein, the term “complex” refers to a composition that comprises an active ingredient or its salt coordinated to at least one resin. By “coordinated” it is meant that at least one atom of the active ingredient or its salt forms a bond with the resin.

As used herein, the term “suspension” refers to a composition that is at least bi-phasic in that it contains a continuous phase and at least one discontinuous phase. The term “suspended” refers to the state of the substance that is in the discontinuous phase of a suspension.

As used herein, the term “steady” refers to a state in which the amount of the drug reaching the system is approximately the same as the amount of the drug leaving the system. Thus, at steady state, the patient's body eliminates the drug at approximately the same rate that the drug becomes available to the patient's system through absorption into the bloodstream.

The modified release liquid pharmaceutical composition of the present invention comprises dextromethorphan, brompheniramine, and pseudoephedrine or pharmaceutically acceptable salt thereof along with one or more rate controlling polymers and pharmaceutically acceptable excipients, wherein the composition provides steady therapeutic levels over 8 to 24 hours after administration.

In an embodiment, the composition provides steady therapeutic levels over 12 hours after administration.

At least one of bromrpheniramine, pseudoephedrine, dextromethorphan or their pharmaceutically acceptable salt in the composition of the present invention is complexed or coated with an ion exchange resin.

In a further embodiment, the modified release liquid pharmaceutical composition comprises dextromethorphan polistirex, brompheniramine polistirex, and pseudoephedrine polistirex along with one or more rate controlling polymers and pharmaceutically acceptable excipients.

The modified release pharmaceutical formulations of the invention exhibit dissolution patterns, which result in the reduction of various side effects normally associated with the use of such drugs. For example, cough/cold formulations containing pseudoephedrine hydrochloride are known to cause central nervous system disorders, such as enhanced agitation and insomnia. Such formulations when used according to the invention show significantly reduced side effects.

The modified release may also be achieved by various techniques known in the art such as use of rate controlling polymers.

In an embodiment, the drug-resin complexes are coated with at least one layer of one or more rate controlling polymers to provide sustained release of drugs for 8 to 24 hours after administration.

Rate controlling polymers include hydrophilic and hydrophobic polymers. Suitable hydrophilic or hydrophobic polymers comprise one or more of polyvinyl acetate, cellulose acetate, cellulose acetate butyrate, cellulose acetate propionate, ethyl cellulose, a fatty acid, a fatty acid ester, an alkyl alcohol, a wax, shellac, rosin, zein (prolamine from corn), povidone, kollidon SR, a poly(meth)acrylate, microcrystalline cellulose or poly(ethylene oxide), polyuronic acid salts, cellulose ethers, xanthan gum, tragacanth gum, gum karaya, guar gum, acacia, gellan gum locust bean gum, alkali metal salts of alginic acid or pectic acid, sodium alginate, potassium alginate, ammonium alginate, hydroxypropyl cellulose, hydroxy ethyl cellulose, hydroxypropyl methyl cellulose, carboxyvinyl polymers, polymerized gelatin, shellac, methacrylic acid copolymer type C NF, cellulose butyrate phthalate, cellulose hydrogen phthalate, cellulose propionate phthalate, polyvinyl acetate phthalate (PVAP), cellulose acetate phthalate (CAP), cellulose acetate trimellitate (CAT), hydroxypropyl methylcellulose phthalate, hydroxypropyl methylcellulose acetate, dioxypropyl methylcellulose succinate, carboxymethyl ethyl cellulose (CMEC), hydroxypropyl methylcellulose acetate succinate (HPMCAS), and acrylic acid polymers and copolymers like methyl acrylate, ethyl acrylate, methyl methacrylate and/or ethyl methacrylate with copolymers of acrylic and methacrylic acid esters (Eudragit NE, Eudragit RL, Eudragit RS).

The taste masking is achieved by complexing the drug with ion exchange resin, complexing agents or by use of pharmaceutically acceptable polymeric materials. In a preferred embodiment, the taste masking is achieved by complexing the drug with ion exchange resin.

The ion-exchange resin comprises one or more of organic, inorganic, anionic or cationic types. Examples of anionic resins include one or more of primary, secondary or tertiary amine functionalities or mixtures thereof put into structures such as epichloro-hydrin-amine condensates and acrylic polymers or styrene divinyl benzene copolymers and the like. Examples of cationic resins include one or more of acrylic or methacrylic acid cross-linked with a difunctional monomer (e.g. divinyl benzene), sulfonated or carboxylated copolymers of styrene-divinyl benzene, etc.

In an embodiment, ion exchange resins suitable for use in the modified release liquid composition are polistirex, sodium polystyrene sulphonate (available commercially under trade name Amberlite®).

Suitable complexing agents may be selected from one or more of cyclodextrins or derivatives thereof such as α-cyclodextrin, β-cyclodextrin, γ-cyclodextrin, hydroxypropyl-α-cyclodextrin, hydroxypropyl-β-cyclodextrin, dimethyl-β-cyclodextrin, 2-hydroxyethyl β-cyclodextrin, trimethyl-β-cyclodextrin, sulfonated cyclodextrins and the like, in anhydrous or hydrated form.

The pharmaceutical composition of the invention further may comprise pharmaceutically acceptable excipients selected from one or more of binders, fillers, disintegrants, glidants, lubricants, surfactants, thickening agents or viscosity modifiers, suspending agent, sweeteners, flavors, colors, solubilizers, stabilizers and preservatives.

Suitable binder may include one or more of, povidone, starch, stearic acid, gums, celluloses, alginic acids, chitosan, chitin, polyethylene glycol and the like.

Suitable fillers may include one or more of saccharose, glucose, fructose, maltose, maltitol, mannitol, dextrins such as maltodextrins; xylitol, sorbitol, microcrystalline cellulose, titanium dioxide, calcium phosphate, calcium sulfate, kaolin, dry starch, powdered sugar, or silicates such as magnesium aluminium silicate.

Suitable disintegrant may include one or more of starch, croscarmellose sodium, crospovidone, or sodium starch glycolate.

Suitable glidant may include one or more of colloidal silicon dioxide, talc or cornstarch.

Suitable lubricant may include one or more of magnesium stearate, zinc stearate, calcium stearate, stearic acid, sodium stearyl fumarate, hydrogenated vegetable oil, or glyceryl behenate.

Suitable surfactants are those known to ordinary skilled in the art and may include one or more of amphoteric, non-ionic, cationic or anionic surfactants. Suitable surfactants comprises one or more of sodium lauryl sulfate, monooleate, monolaurate, monopalmitate, monostearate or another ester of polyoxyethylene sorbitane, sodium dioctylsulfosuccinate (DOSS), lecithin, stearylic alcohol, cetostearylic alcohol, cholesterol, polyoxyethylene ricin oil, polyoxyethylene fatty acid glycerides, poloxamer, or cremophore RH 40.

Suitable thickening agents or viscosity modifiers may include one or more of methylcellulose, carboxymethylcellulose, microcrystalline cellulose, ethylcellulose, hydroxyethylcellulose, hydroxypropylcellulose, hydroxypropylmethylcellulose, alginate, carageenan, xanthan gum, acacia, tragacanth, locust bean gum, guar gum, carboxypolymethylene, polyvinyl pyrrolidone, polyvinyl alcohol, poloxamer, magnesium aluminum silicate (veegum), bentonite, hectorite, povidone, maltitol, chitosan or combination thereof.

Suitable sweetener may include one or more of monosaccharides, disaccharides and polysaccharides, e.g. xylose, ribose, glucose, mannose, galactose, fructose, sucrose, maltose, invert sugar, partially hydrolyzed starch, corn syrup solids, mannitol, xylitol, D-sorbitol, erythritol, pentitol, hexitol, malitol, dihydrochalcones, monellin, steviosides or glycyrrhizin; saccharin in free acid form, soluble saccharin salts, e.g. sodium or calcium saccharin salts, cyclamate salts or acesulfame K; dipeptide based sweeteners, such as L-aspartic acid derived sweeteners, e.g. aspartame; water-soluble sweeteners derived from naturally occurring water-soluble sweeteners, e.g. sucralose; or protein based sweeteners, e.g. thaumatococcus danielli (Thaumatin I and II).

Suitable flavoring agents may include those known to the skilled artisan, such as natural, “natural-like” and artificial flavors. These flavors may be chosen e.g. from synthetic flavor oils, flavoring aromatics, oleo-resins and extracts derived e.g. from plants, leaves, flowers or fruits.

Preservatives may include one or more of sodium benzoate, sorbates, such as potassium sorbate, salts of edetate (also known as salts of ethylenediaminetetraacetic acid or EDTA, such as disodium edetate), benzaldionium chloride, or parabens.

The formulations of the invention optionally include one or more stabilizing agents to increase the stability and/or compatibility of the suspension when formulated into a dosage form. Suitable stabilizing agents are suspending agents, flocculating agents, thickening agents, gelling agents, buffering agents, antioxidants, preservatives, antimicrobial agents, and mixtures thereof. Ideally, the agent acts to minimize irreversible aggregation of suspended particles, and to maintain proper flow characteristics to ease manufacturing processes, e.g., to ensure that the formulation can be readily pumped and filled into desired container.

Suspending agents may include one or more from cellulose derivatives, clays, natural gums, synthetic gums, or other agents known in the art. Specific suspending agents, by way of example, include microcrystalline cellulose, sodium carboxymethylcellulose, powdered cellulose, ethymethylcellulose, hydroyxypropyl methylcellulose, methylcellulose, ethylcellulose, ethylhydroxy ethylcellulose, hydroxypropyl cellulose, attapulgite, bentonite, hectorite, montmorillonite, silica gel, fumed silicon dioxide, colloidal silicon dioxide, acacia, agar, carrageenan, guar gum, locust bean gum, pectin, sodium alginate, propylene glycol alginate, tamarind gum, xanthan gum, carbomer, povidone, sodium starch glycolate, starches, tragacanth, magnesium aluminum silicate, aluminum silicate, magnesium silicate, gelatin, or glycyrrhizin. These suspending agents can further impart different flow properties to the suspension. The flow properties of the suspension can be Newtonian, plastic, pseudoplastic, thixotropic or combinations thereof. Mixtures of suspending agents may also be used to optimize flow properties and viscosity.

Suitable buffering agents may include but not limited to one or more of a bicarbonate salt of a Group IA metal, an alkali earth metal buffering agent, a calcium buffering agent, a magnesium buffering agent, an aluminum buffering agent and the like, sodium bicarbonate, potassium bicarbonate, magnesium hydroxide, magnesium lactate, magnesium gluconate, magnesium oxide, magnesium aluminate, magnesium carbonate, magnesium silicate, magnesium citrate, aluminum hydroxide, aluminum phosphate, aluminum hydroxide/magnesium carbonate, potassium carbonate, potassium citrate, aluminum hydroxide/sodium bicarbonate coprecipitate, aluminum glycinate, aluminum magnesium hydroxide, sodium citrate, sodium tartrate, sodium acetate, sodium carbonate, sodium (polyphosphate, sodium dihydrogen phosphate, potassium polyphosphate, sodium pyrophosphate, potassium pyrophosphate, disodium hydrogenphosphate, dipotassium hydrogenphosphate, trisodium phosphate, tripotassium phosphate, potassium metaphosphate, calcium acetate, calcium glycerophosphate, calcium chloride, calcium hydroxide, calcium lactate, calcium carbonate, calcium gluconate, calcium bicarbonate, calcium citrate, calcium phosphate magnesium phosphate, potassium phosphate, sodium phosphate, trihydroxymethylaminomethane, ail amino acid, an acid salt of an amino acid, an alkali salt of an amino acid, and combinations of any of the foregoing.

Moreover, the composition of the invention optionally include usual auxiliaries known in the art such as saliva stimulating agents like citric acid, lactic acid, malic acid, succinic acid, ascorbic acid, adipic acid, fumaric acid, tartaric acids; cooling sensation agents like maltitol, monomenthyl succinate, ultracool; stabilizers like gums, agar; taste masking agents like acrylic polymers, copolymers of acrylates, celluloses, resins; coloring agents like titanium dioxide, natural food colors, dyes suitable for food, drug and cosmetic applications; preservatives like alpha-tocopherol, citric acid, butylated hydroxytoluene, butylated hydroxyanisole, ascorbic acid, fumaric acid, malic acid, sodium ascorbate or ascorbic acid palmitate or effervescing agents like citric acid, tartaric acid, sodium bicarbonate, or sodium carbonate.

The liquid pharmaceutical composition of the present invention can be prepared by the various processes known in the art.

The invention is further illustrated by the following examples which are provided merely to be exemplary of the invention and do not limit the scope of the invention. Certain modifications and equivalents will be apparent to those skilled in the art and are intended to be included within the scope of the invention.

### Example 1

### Preparation of Dextromethorphan ER/Pseudoephedrine HCl ER/Brompheniramine Meleate ER Suspension

TABLE 1

| Sr. No. | Ingredients | mg/5 mL |
|---|---|---|
| 1 | Extended release coated Dextromethorphan | 75-95 |
| | Polistirex | |
| | [Dextromethrophan HBr as Coated drug resin | |
| | beads eq. to 30 mg Dextromethorphan HBr | |
| | (monohydrate) | |
| 2 | Extended release coated Pseudoephedrine | 325-345 |
| | HCl | |
| | Amberlite IRP 69 Drug Resinate | |
| | [Pseudoephedrine HCl as Coated drug resin | |
| | beads eq. to 60 mg Pseudoephedrine HCl] | |
| 3 | Extended release coated Brompheniramine | 15-25 |
| | Meleate | |
| | Amberlite IRP 69 Drug Resinate | |
| | [Brompheniramine Meleate as Coated drug | |
| | resin beads eq. to 6 mg Pseudoephedrine HCl] | |
| 4 | Trisodium citrate dihydrate Emprove ® | 1-10 |
| 5 | Citric acid monohydrate Emprove ® | 7-15 |
| 6 | High Fructose Corn Syrup (Hisweet-55 HFCS | 820-860 |
| | 55) | |
| 7 | Pharma grade sugar as 50% w/w liquid (in | 1400-1600 |
| | purified water) | |
| 8 | Propylene glycol | 40-60 |
| 9 | Butylhydroxyanisole | 0.5-2 |
| 10 | Butylhydroxytoluene | 0.1-1 |
| 11 | Methyl paraben | 5-15 |
| 12 | Propyl paraben | 0.5-2 |
| 13 | Xanthan Gum (Xantural 75) | 5-15 |
| 14 | Tragacanth | 15-35 |
| 15 | Polysorbate 80 (Crillet 4HP) | 0.1-2 |
| 16 | FD&C yellow no. 6 | 0.01-0.5 |
| 17 | Orange flavour 501202T* | 0.01-0.5 |
| 18 | Purified water | qs to 5 mL |

Procedure:

Trisodium citrate dihydrate and citric acid monohydrate were weighed and dissolved in purified water. In the final mixing tank High fructose corn syrup was weighed and to this citrate buffer and liquid sugar were added under stirring. Butylhydroxyanisole, butylhydroxytoluene, methyl paraben and propyl paraben were added to propylene glycol under gentle heating to dissolve and then subsequently cooled. Tragacanth and xanthan gum was added to above cooled solution. Wetted gum solution of was transferred to buffered sugar-High fructose corn syrup solution under stirring. FD&C yellow no. 6 was added to 2.5% of total quantity of purified water under gentle stirring. Weighed quantity of flavor was added to suspension base. In remaining 5-10% of total volume polysorbate 80 was added and stirred, lubricated extended release coated Dextromethorphan HBr-resinate, Pseudoephedrine HCl-resinate and Brompheniramine Meleate-resinate were wetted. Make up the desired volume; Volume of suspension is calculated based on the density of the base or through some pre calibrated measuring stick.

### Example 2

### Preparation of Extended Release Dextromethorphan Polistirex

TABLE 2

| | | |
|---|---|---|
| Sr. No. | Ingredients | Qty (mg/dose^{$}) |

Resin Pretreatment

| | | |
|---|---|---|
| 1 | Sodium polystyrene sulphonate | 25-45 |
| | (Amberlite IRP 69)^{$} | |

Washing of Resins

| | | |
|---|---|---|
| 2 | Citric acid monohydrate* | 1-10 |
| 3 | Trisodium Citrate dihydrate* | 1-4 |
| 4 | Purified Water* | 0.1-1 |

BHT Treatment

| | | |
|---|---|---|
| 5 | BHT* | 0.1-1 |
| 6 | Propylene Glycol* | 10-20 |

Drug Complexation

| | | |
|---|---|---|
| 7 | Dextromethorphan HBr | 20-40 |
| | (monohydrate) USP | |
| 8 | Purified Water* | 250-300 |

PEG treatment

| | | |
|---|---|---|
| 9 | PEG 3350 | 2-6 |
| 10 | Purified Water* | 25-35 |

ER Coating^{@}

| | | |
|---|---|---|
| 11 | Eudragit NE 30D | 10-15 |
| 12 | Glyceryl monostearate | 0.1-2 |
| 13 | Simethicone | 0.1-1 |
| 14 | Talc | 1-5 |
| 15 | Purified Water* | 40-60 |

Lubrication^{$}

| | | |
|---|---|---|
| 16 | Talc | 1-5 |

*Doesn't remain in the final product-removed while washing and drying.

Procedure:

Resins were sifted. Initial LOD was recorded. Citric acid monohydrate and Trisodium citrate dihydrate was dissolved in purified water. The washed resins were subjected to three cycles washing with buffer and one cycle with purified water. After each cycle, water/buffer was drained out with minimum loss of resins. Washed resins were subsequently dried. Final LOD required was 4-7.5% w/w.

BHT Dispersion was prepared by dissolving BHT in propylene glycol under gentle heating. Washed resin taken on anhydrous basis was then dispersed in suitable tank containing deionised water under stirring. BHT dispersion was then added to slurry, following which the free uncomplexed is determined after standing for 12-16 Hrs. Washing was done to remove uncomplexed drug. After each cycle, water was drained out with minimum loss of resins. Drug resin complex were subsequently dried. Dried beads were then sifted.

PEG 3350 was dispersed in purified water under stirring. PEG impregnated beads were carried out and subsequently dried. Dried beads were then sifted.

Glyceryl monostearate (GMS) was weighed along with simethicone suspension; this was added under continuous stirring to deionised water. The above suspension was then homogenized in a homogenizer. Talc was dispersed in water under stirring and was added to suspension of GMS under homogenization. Eudragit NE 30D was separately weighed out and the talc-GMS dispersion was added slowly and continuously to the Eudragit solution. ER coating of PEG impregnated beads were carried out in Glatt.

### Example 3

### Preparation of Extended Release Coated Pseudoephedrine HCl-Amberlite IRP 69 Drug Resinate

TABLE 3

| | | |
|---|---|---|
| Sr. No. | Ingredients | Qty (mg/dose^{$}) |

Resin Pretreatment

| | | |
|---|---|---|
| 1 | Sodium polystyrene sulphonate | 125-145 |
| | (Amberlite IRP 69)^{$} | |

Washing of Resins

| | | |
|---|---|---|
| 2 | Citric acid monohydrate* | 15-25 |
| 3 | Trisodium Citrate dihydrate* | 5-15 |
| 4 | Purified Water* | 0.1-1 |

BHT Treatment

| | | |
|---|---|---|
| 5 | BHT* | 0.1-2 |
| 6 | Propylene Glycol* | 40-60 |

Drug Complexation

| | | |
|---|---|---|
| 7 | Pseudoephedrine HCl | 80-100 |
| 8 | Purified Water* | 750-850 |

PEG treatment

| | | |
|---|---|---|
| 9 | PEG 4000 | 45-65 |
| 10 | Purified Water* | 55-65 |

EC Coating^{@}

| | | |
|---|---|---|
| 11 | Ethocel 45C | 30-40 |
| 12 | Triethyl citrate | 2-8 |
| 13 | Talc | 10-20 |
| 14 | IPA: DCM (60:40)* | 2800-2850 |

*Doesn't remain in the final product- removed while washing and drying.

Procedure:

Resins were sifted. Initial LOD was recorded. Citric acid monohydrate and Trisodium citrate dihydrate was dissolved in purified water. The washed resin were subjected to three cycle washing with buffer and one cycle with purified water. After each cycle, water/buffer was drained out with minimum loss of resins. Washed resins were subsequently dried. Final LOD required was 4-7.5% w/w.

BHT Dispersion was prepared by dissolving BHT in propylene glycol under gentle heating. Washed resin taken on anhydrous basis was then dispersed in suitable tank containing deionised water under stirring. BHT dispersion was then added to slurry, following which the free uncomplexed is determined after standing for 12-16 Hrs. Washing was done to remove uncomplexed drug. After each cycle, water was drained out with minimum loss of resins. Drug resin complex were subsequently dried. Dried beads were then sifted.

PEG 3350 was dispersed in purified water under stirring. PEG impregnated beads were carried out and subsequently dried. Dried beads were then sifted.

Required quantity of IPA:DCM (60:40) was mixed. Ethocel 45 was weighed and added in the container and stirred. Triethyl citrate was weighed and mixed in the container and stirred. Talc was weighed and mixed in the container and stirred. ER coating of PEG impregnated beads were carried out in Glatt.

### Example 4

### Preparation of Extended Release Coated Brompheniramine Meleate Polistirex

TABLE 4

| | | |
|---|---|---|
| S. No. | Qty (mg/dose^{$}) | Qty (mg/dose^{$}) |

Resin Pretreatment

| | | |
|---|---|---|
| 1 | Sodium polystyrene sulphonate | 5-10 |
| | (Amberlite IRP 69)^{$} | |

Washing of Resins

| | | |
|---|---|---|
| 2 | Citric acid monohydrate* | 0.1-2 |
| 3 | Trisodium Citrate dihydrate* | 0.1-2 |
| 4 | Purified Water* | 0.01-0.5 |

BHT Treatment

| | | |
|---|---|---|
| 5 | BHT* | 0.01-0.5 |
| 6 | Propylene Glycol* | 1-5 |

Drug Complexation

| | | |
|---|---|---|
| 7 | Brompheniramine meleate | 1-10 |
| 8 | Purified Water* | 40-70 |

PEG treatment

| | | |
|---|---|---|
| 9 | PEG 4000 | 1-5 |
| 10 | Purified Water* | 20-40 |

EC Coating^{@}

| | | |
|---|---|---|
| 11 | Eudragit NE 30D | 2-8 |
| 12 | Glyceryl monostearate | 0.1-1 |
| 13 | Simethicone | 0.01-0.5 |
| 14 | Talc | 0.5-2 |
| 15 | Purified Water* | 15-20 |

Procedure:

Resins were sifted. Initial LOD was recorded. Citric acid monohydrate and Trisodium citrate dihydrate was dissolved in purified water. The washed resins were subjected to three cycle washing with buffer and one cycle with purified water. After each cycle, water/buffer was drained out with minimum loss of resins. Washed resins were subsequently dried. Final LOD required was 4-7.5% w/w.

BHT Dispersion was prepared by dissolving BHT in propylene glycol under gentle heating. Washed resin taken on anhydrous basis was then dispersed in suitable tank containing deionised water under stirring. BHT dispersion was then added to slurry, following which the free uncomplexed is determined after standing for 12-16 Hrs. Washing was done to remove uncomplexed drug. After each cycle, water was drained out with minimum loss of resins. Drug resin complex were subsequently dried. Dried beads were then sifted.

PEG 3350 was dispersed in purified water under stirring. PEG impregnated beads were carried out and subsequently dried. Dried beads were then sifted.

Glyceryl monostearate (GMS) was weighed along with simethicone suspension; this was added under continuous stirring to deionised water. The above suspension was then homogenized in a homogenizer. Talc was dispersed in water under stirring and was added to suspension of GMS under homogenization. Eudragit NE 30D was separately weighed out and the talc-GMS dispersion was added slowly and continuously to the Eudragit solution. ER coating of PEG impregnated beads were carried out in Glatt.

We claim:

1. A modified release liquid pharmaceutical composition comprising:
a. brompheniramine or pharmaceutically acceptable salt thereof;
b. pseudoephedrine or pharmaceutically acceptable salt thereof;
c. dextromethorphan or pharmaceutically acceptable salt thereof complexed or coated with one or more an ion exchange resins,
wherein the composition exhibits a steady therapeutic levels over 8 to 24 hours after administration.

2. The modified release liquid pharmaceutical composition of claim 1, wherein pseudoephedrine or salt thereof is complexed or coated with one or more an ion exchange resins.

3. The modified release liquid pharmaceutical composition of claim 1, wherein dextromethorphan or salt thereof is complexed or coated with one or more an ion exchange resins.

4. The modified release liquid pharmaceutical composition of claim 1, 2, or 3, wherein the ion exchange resin comprises one or more organic, inorganic, anionic, and cationic exchange resins.

5. The modified release liquid pharmaceutical composition of claim 4, wherein the ion exchange resin comprises polistirex, sodium polystyrene sulphonate, and mixture thereof.

6. The modified release liquid pharmaceutical composition of claim 1, wherein the composition comprises one or more rate-controlling polymers.

7. The modified release liquid pharmaceutical composition of claim 6, wherein the rate-controlling polymers comprises one or more hydrophilic polymers, hydrophobic polymers, or mixture thereof.

8. An aqueous suspension comprising:
a. brompheniramine or pharmaceutically acceptable salt or pharmaceutically acceptable salt thereof complexed or coated with sodium polystyrene sulphonate resin;
b. pseudoephedrine or pharmaceutically acceptable salt or ion exchange resin thereof complexed or coated with sodium polystyrene sulphonate resin;
c. dextromethorphan or pharmaceutically acceptable salt or ion exchange resin thereof complexed or coated with polistirex resin;
d. one or more rate-controlling polymers,
wherein the composition provides steady therapeutic levels over 8 to 24 hours after administration.

9. A method of treating one or more symptoms selected from upper respiratory tract infection, common cold, and allergic rhinitis, which method comprises of administering the modified release liquid pharmaceutical composition 1 or 8 to a patient in need thereof.

10. A taste masked modified release liquid pharmaceutical composition comprising:
a. brompheniramine or pharmaceutically acceptable salt thereof complexed or coated with an ion exchange resin;
b. pseudoephedrine or pharmaceutically acceptable salt thereof complexed or coated with an ion exchange resin;
c. dextromethorphan or pharmaceutically acceptable salt thereof complexed or coated with an ion exchange resin,
wherein the composition provides steady therapeutic levels over 8 to 24 hours after administration.
